(19) United States
(12) Patent Application Publication
(10) Pub. No.: US 2023/0029661 A1
(43) Pub. Date: Feb. 2, 2023

(54) DIRECT LASER TRABECULOPLASTY METHOD AND APPARATUS

(71) Applicant: Ellex Medical Pty Ltd, Mawson Lakes, South Australia (AU)

(72) Inventors: Eric Benson, Magill, South Australia (AU); David Haarhoff, Highbury, South Australia (AU)

(21) Appl. No.: 17/759,069

(22) PCT Filed: Feb. 8, 2021
(86) PCT No.: PCT/AU2021/050102

§ 371 Date: Jul. 19, 2022

(30) Foreign Application Priority Data
Feb. 7, 2020 (AU) 2020900344

(51) Int. Cl. A61F 9/008

(52) U.S. Cl. CPC A61F 9/008; A61F 2009/00851; A61F 2009/00891

(57) ABSTRACT

Apparatus and methods for treating glaucoma in a patient's eye (25) are provided. A treatment laser beam is directed at the trabecular meshwork of the patient's eye to initiate reactions that promote improved drainage of aqueous humour fluid.

### FIELD OF INVENTION

This invention relates to an ophthalmic treatment of human eyes and more specifically to the treatment of glaucoma using a laser beam, whereby it is directed at the trabecular meshwork to initiate reactions that promote improved drainage of aqueous humour fluid.

The invention relates to an ophthalmic apparatus for treating glaucoma in a patient's eye.

The invention relates further to a method of treating glaucoma.

### BACKGROUND

Glaucoma is a disease in which vision is impaired as a result of damage to the optic nerve or retina, and is responsible for about 25% of blindness in developed countries. A common contributor to this damage is elevated pressure of the fluid, known as the aqueous humour, within the eye. The increased intra-ocular pressure causes progressive death of retinal ganglion cells and damages axons that transfer visual information to a brain via an optic nerve. The aqueous fluid is constantly and slowly replaced by the body with the ingress coming from a ciliary body just beneath the iris and a balancing drainage taking place through an annular spongy tissue, known as a trabecular meshwork around the edge of the iris where it meets the cornea which transitions into the sclera. Drainage takes place from the meshwork through to a structure called Schlemm's canal and eventually into the body's circulatory system.

The primary cause of elevated pressure in an eye is due to an imbalance between the ingress and egress of fluid due to malfunctioning of the annular trabecular meshwork. This meshwork provides drainage of the fluid through ducts that are distributed around the trabecular annulus, however with age these ducts become blocked with cellular debris. Methods to improve the drainage have hitherto been attempted with either medication or surgical means. A more recent method, known as laser trabeculoplasty, relies on directing a pulsed, focused laser beam onto the trabecular meshwork with sufficient intensity that pigmented melanin cells suffer damage and initiate biological changes whereby laser-damaged sites are repopulated by cells from a non-filtering region of the trabecular meshwork. These have been found to serve as stem cells producing fresh and functioning cells that have been found to restore the drainage by the trabecular meshwork.

Currently, delivery of a laser beam to the trabecular meshwork is achieved by directing a laser beam obliquely through the cornea of an eye with the aid of an optical element placed in contact with the eye, the element including a mirror to direct the laser beam sideways to the trabecular meshwork. This treatment method is known as selective laser trabeculoplasty or SLT. With this system an ophthalmic practitioner is required to rotate the optical element to treat multiple regions around the trabecular meshwork. When sufficient intensity is achieved, the reaction can be identified by production of micro-bubbles. It is the production of microbubbles that the detection of which indicates that the treatment laser energy is sufficient.

The shortcomings of this method are several-fold: It can be difficult for a practitioner to accurately direct the beam to a desired spot on the trabecular meshwork (TM); the procedure requires great skill to avoid risks of injury and infection; and lastly the procedure can be quite lengthy thereby causing discomfort to the patient.

An improvement to the technique has been proposed in a patent application by Belkin (US 2015 0 366 706 A1) in which a treatment laser beam is directed at the trabecular meshwork through the sclera. The deficiency in this method is that neither the ideal dose nor the exact location of a TM is known, both of these parameters are assumed in the application whereas in reality the exact position of the TM is unknown and the required energy dose is speculative. The position or diameter of the TM relative to the iris varies between individuals and generally it cannot be seen through a sclera. The intensity of beam necessary to induce damage to the melanin cells cannot be readily determined as it depends on scattering and absorption in a sclera, the extent of which varies between individuals and on the location along the trabecular meshwork.

### OBJECT OF INVENTION

The object of this invention is to provide a method or apparatus for treating a trabecular meshwork in an eye in a fashion so that the treatment laser beam location and intensity has the potential to be automatically controlled and an operator has minimal involvement with the procedure after it has been initiated.

This objective is achieved by delivering a treatment laser beam through the sclera of an eye at a location and with an energy dose that is determined by detecting and analysing a back-scattered reflection of light from a probe light beam directed at one or more regions near the trabecular meshwork.

This invention provides an uncomplicated means to perform a selective laser trabeculoplasty procedure through a sclera from the front of an eye without any optics contacting the eye.

Furthermore, in the broadest sense, the position of the trabecular meshwork does not need to be known accurately, though a method described below allows a sufficiently accurate method of determining its position.

This invention can be further described as a method or apparatus for treating glaucoma whereby a pulsed treatment laser beam is focused behind the sclera and through to a trabecular meshwork of an eye, the method characterised by including a probe beam of light coupled to an optical coherence tomography sub-system that both identifies the location of the meshwork and detects when micro-bubbles have been formed during a phase in which the energy of the treatment laser beam is increased.

In another form, this invention can be described as a method or apparatus for treating glaucoma whereby a treatment pulsed laser beam is focused behind the sclera and through to a trabecular meshwork of an eye, the method characterised by sequentially projecting, using scanning means, a multitude of segmented lines spanning between an inner and outer radii (with reference to the centre of an iris) and the laser beam energy being set to be sufficient to cause damage to the melanin cells in the trabecular meshwork.

The treatment laser beam energy is determined by performing an initial test in which the energy is increased after each radial scan until micro-bubbles are formed, the formation being detected by a change in the backscattered intensity of a probe beam of light, the change resulting from the gas-liquid interfaces that are generated.

The laser probe beam can either be the same as the treatment beam or a separate laser beam of more optimal wavelength and intensity.

In an alternative form of the invention, the location of the TM is determined in a multitude, such as 8 or 16 or more, radial locations and an interpolation performed to determine its location at different radial orientations. The locations are recorded with reference to the outer diameter of the iris that is subsequently used as a reference location using digital imaging means.

In this description, it is recognised that a treatment beam will not focus sharply behind the sclera on account of scattering in the translucent tissue, nevertheless it is this condition that will be referred to as one in which a beam is focussed.

With regard to an additional aspect of the invention the task of the invention is solved by an ophthalmic apparatus for treating glaucoma in a patient's eye comprising a treatment laser module delivering a treatment laser beam, and comprising a detection system for detecting micro-cavitation, in particular micro-bubbles, formed on account of the treatment laser beam in the patient's eye, in particular at the trabecular meshwork of the patient's eye.

If the location, and/or the time, and/or the levels of micro-cavitation respectively of micro-bubbles is better known, it is possible to minimise the energy delivered to the eye and to minimise the duration of the treatment.

Insofar the detection system allows an additional position control to control the position of the micro-cavitation at the trabecular meshwork.

This alone is an advantageous further development of existing methods for the treatment of glaucoma.

In regard to a further aspect of the invention the present task at hand is solved by an ophthalmic apparatus for treating glaucoma in a patient's eye comprising a treatment laser module delivering a treatment laser beam, and comprising a detection system for detecting the location in 2 or 3 dimensions and/or the shape, in particular a possible asymmetry, of the trabecular meshwork of the patient's eye.

If the location and/or the shape is better known before treatment, it is possible to minimise the energy delivered to the eye and to minimise the duration of the treatment.

Therefore, on the one hand the detection system can be equipped with an additional position control to detect the position of the trabecular meshwork and its shape, preferably before activation the treatment laser module.

Thus, the setup can also comprise an ophthalmic apparatus for measuring the eye, in particular the trabecular meshwork of the eye.

On the other hand, the detection system allows for a position control to control the position of the micro-cavitation.

This alone is an advantageous further development of existing methods for the treatment of glaucoma.

It is also possible to correct the position of the micro-cavitation, preferably live during the treatment.

In both cases it is possible to modulate the treatment laser beam in dependence of the information provided by the detection system.

The detection system may be constructed in different ways.

Constructively simple yet precise solutions can be realised if the detection system comprises a tomography system for detecting micro-cavitation.

Cumulatively or alternatively the detection system can comprise an optical coherence tomography (OCT) system for detecting the location and/or the shape, especially a possible asymmetry and/or micro-cavitation.

The detection system can comprise more components, like a camera, a controller, a processor, a scanner or the like.

It is further advantageous, if the apparatus comprising an eye-probe sub-system emits a co-axial probe beam. This allows the treatment site in the eye to be observed particularly well.

If beams focused behind the sclera of a patient's eye and through to a trabecular meshwork of a patient's eye, contactless treatment of the eye can be carried out without any problems.

The task of the invention is additionally fulfilled by an ophthalmic apparatus for treating glaucoma comprising a treatment laser module delivering a treatment laser beam to a scanner and an objective focusing lens, and comprising a co-axial probe beam emitted from an eye-probe sub-system, said beams focused behind the sclera and through to a trabecular meshwork of a patient's eye, the apparatus including a detector preferably within the eye-probe sub-system that senses backscattered light from the probe beam and detects the formation of micro-bubbles formed on account of the treatment laser beam inducing damage to the melanin cells in the trabecular meshwork.

This solution describes a concrete possible apparatus with which the invention can be well realised. Especially, it is possible to minimise the energy delivered to the eye and to minimise the duration of the treatment, as well.

Furthermore, it is particularly advantageous, if the apparatus comprises an energy control system, which modulates the treatment laser beam in dependence on information of the detection system. In this case, the required energy for the treatment laser beam can depend on the formation of micro-cavitation or micro-bubbles, and/or on the shape of the areas of the eye to be treated, in particular the trabecular meshwork.

In particular, with a suitably designed energy control system, the intensity, the duration or the like of the energy level of the treatment laser beam can be determined in dependence on the onset, the progress, the intensity or the like of a formation of micro-cavitation or micro-bubbles.

With regard to an alternative construction method, it is advantageous, if an eye-probe sub-system comprises an optical coherence tomography (OCT) system that further determines the location of the trabecular meshwork prior to delivery of the treatment laser beam. This allows the apparatus to be realised in a structurally simple way.

If an eye-probe sub-system comprises a photo-detector, the observation of specific treatment areas of the eye can be performed more compactly.

It is understood that the treatment laser beam and the probe beam can be provided independently from each other. If the probe beam is identical with the treatment laser beam, it is possible to further simplify the construction of the apparatus.

A particularly robust and error-free design with regard to the laser beams used can be achieved, if the wavelength of the treatment laser beam is in the absorption range of melanin cells and the probe beam is infra-red.

With regards to a further aspect of the invention the present task is solved by a method for treating glaucoma characterised by determining through a sclera the location and/or the shape of a trabecular meshwork and delivering a treatment laser beam to that location with a beam's energy sufficient to generate micro-bubbles. This results in a particularly locally precise treatment, whereby the energy required to generate the micro-bubbles can be set extremely precisely.

Hereby it is possible to minimise the energy delivered to the eye and to minimise the duration of the treatment.

In a very advantageous version of the process the energy is controlled and adjusted depending on the effect of micro-bubbles and/or the size of the micro-cavitation, and/or of the shape and/or location of the trabecular meshwork.

Furthermore, it is advantageous, if an optical coherence tomography system in a first step is used to identify the location of the trabecular meshwork, whereupon the treatment laser beam is directed at that location and either a preset laser energy dose is delivered to the location or the energy dose is increased until the tomography system detects micro-cavitation. This allows the treatment site on the eye to be determined particularly precisely and then to be processed particularly gently with a suitably intensive laser beam.

A particularly preferred process variant provides that the beams follow a pattern in accordance with inputs from an energy control system, in particular a processor and controller. In this way it can be particularly advantageous to ensure that only enough energy can be applied to the treatment area until the micro-bubbles are formed, which indicates sufficient treatment of the trabecular meshwork.

Also, it is advantageous, that the pattern comprises radial lines or radial segments extending from an inner radius R1 to an outer radius R2, the radii corresponding to extremes of the likely position of a trabecular meshwork. This ensures that only those areas of the eye are treated with the treatment laser beam, which are absolutely necessary for the treatment of glaucoma.

At this point, it is also claimed that the described methods can also be supplemented by further technical features described herein, in particular by features of the apparatus, in order to advantageously further develop the methods or to be able to represent or formulate method specifications even more precisely.

Here it may be explicitly noted, that any characteristics of the previous figures and/or claims may be combined if desired to combine and accomplish the effects, characteristics and advantages cumulatively.

Naturally, the previously mentioned examples of embodiment are only first design of the invention. Therefore, the embodiment of the invention is not restricted to these variants.

All described characteristics in the application are claimed as essential to the invention as long as they are novel in relation to the state of art, either on their own or in any possible combination.

### DESCRIPTION OF INVENTION

The invention can be better understood by describing two preferred embodiments illustrated in the accompanying figures in which:

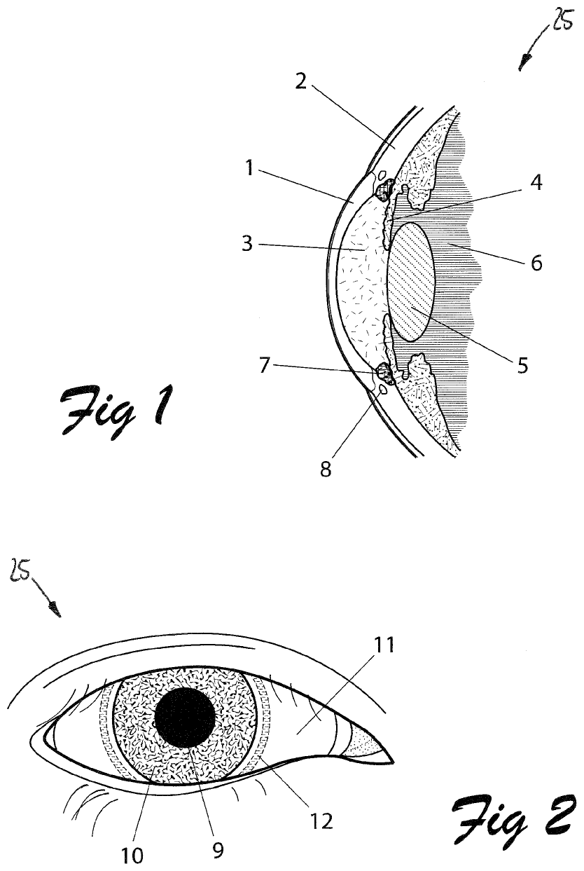

FIG. 1 shows a cross-section of an eye featuring a trabecular meshwork.

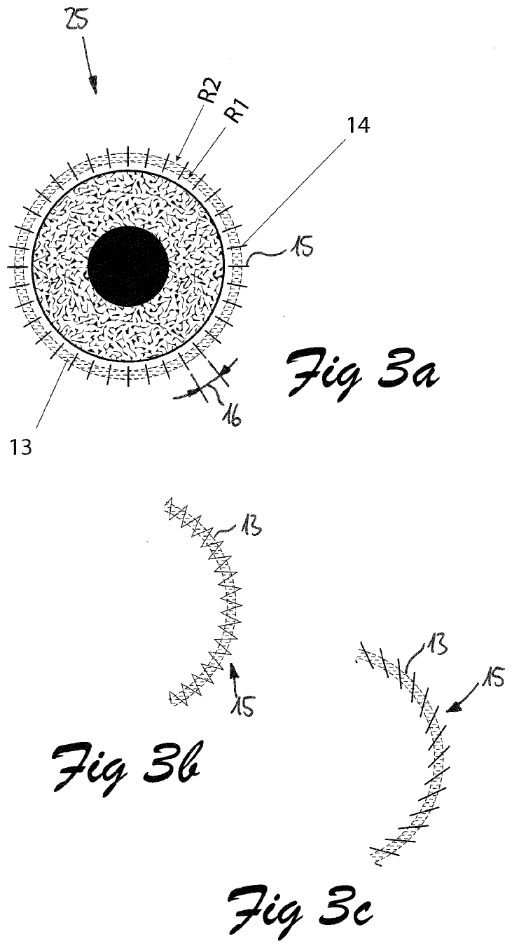

FIG. 2 shows a front-on view of an eye

FIG. 3 shows possible laser spot patterns projected onto an eye.

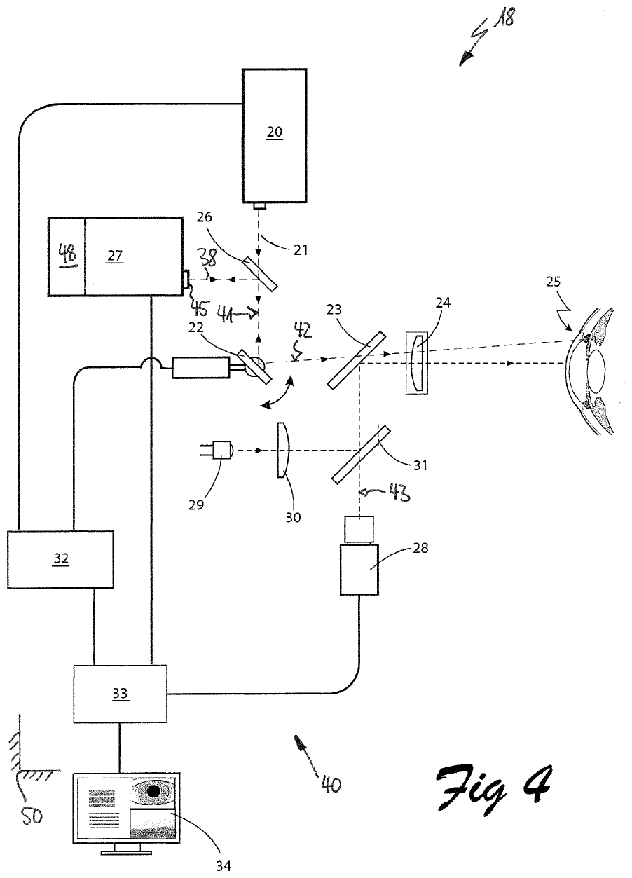

FIG. 4 shows a schematic of a preferred embodiment of the invention.

Referring to FIG. 1, a cornea 1 of a left eye 25 of a patient (not shown) connects to a sclera 2. The fluid-filled anterior chamber 3 is contained by the pigment epithelium or iris 4 that surrounds the lens 5. The posterior chamber 6 contains vitreous humor and represents the largest volume of an eye. Nestled between the outer edges of the cornea and the iris is the trabecular meshwork 7, through which drainage is effected into Schlem's canal 8. The trabecular meshwork 7 has a triangular cross-section.

FIG. 2 shows a left eye 25 as it might be presented to a practitioner. It shows a pupil 9 surrounded by an iris 10. The adjacent white sclera 11 conceals a trabecular meshwork 12, shown having exaggerated width within dashed lines.

The width of this meshwork 7 or 12 is typically in the order of 350 microns with a depth of 50-150 microns.

In one preferred embodiment of the invention a system delivers a probe beam 38 (cf. FIG. 4) of light that is scanned in a pattern as shown in FIG. 3a.

Referring to the FIG. 3a, a trabecular meshwork 13 lies within an ‘uncertainty annulus’ 14 having an inner radius R1 and an outer radius R2.

A probe beam traverses along short radial lines 15 between the inner and outer radius R1 and R2, repeating for different orientations around the eye 25 separated by some angle 16 of about 1-10 degrees.

The choice of angle 16 being a compromise between treatment duration and sufficient density of trabecular tissue damage.

While radial lines 15 are shown, other options are possible that traverse between an inner and outer radius R1 and R2 such as a zig-zag segment 15 in FIG. 3b following a circular path or angled radial lines 15 such as in FIG. 3c.

Curved forms of these patterns could also be used.

While the term ‘lines’ has been used, this refers to the path of the beam even though on a microscopic level the reactive path comprises discrete spots corresponding to the digitized location of the probe beam 38 arranged in a line.

The pattern is generated by a conventional galvanometer two-axis scanner 22 (cf. FIG. 4) or other devices.

Another key aspect of the treatment is determining the energy necessary to achieve damage to the melanin cells in the trabecular meshwork 7, 12 resp. 13.

It has been recognised with conventional SLT that one way to ensure damage has been achieved is to increase the energy until a vapour gas bubble is formed within the meshwork 7, 12 resp. 13.

Conventionally this occurrence is observed by a practitioner, however with this invention the production of micro-bubbles is detected by a change in the backscattered reflection of an observation or treatment laser beam 21 (cf. FIG. 4), though preferably an observation beam.

FIG. 4 illustrates a schematic of a first possible embodiment of the optical arrangement to achieve delivery of a treatment laser beam 21 and bubble detection.

Referring to the FIG. 4, a treatment laser module 20 generates an input laser treatment beam 21.

This treatment laser module 20 includes any necessary attenuators, beam conditioners and shutters (not shown separately).

The laser treatment beam 21 exiting the treatment laser module 20 is directed at a 2-axis scanner 22 that transmits through a dichroic or partial reflector 23 and through a focusing lens 24 and onto an eye 25.

An eye-probe system 27 comprises a probe beam of light 38 and a detection system 40 that will be discussed in more detail below.

The light, especially the probe beam 38, leaving and entering the eye-probe system 27 has an optical path 41 that is substantially coincident with the treatment laser beam path 42, with a combination of the paths 41 and 42 achieved with reflector 26.

This reflector 26 is preferably a dichroic mirror (not referenced additional) tailored for the reflecting and transmitting wavelengths concerned.

To position and monitor the eye 25, a camera 28 captures light reflected off the reflector 23.

This camera 28 also provides an image which can be used to determine a scan pattern and to provide a record for future reference.

To assist in minimising movement of a patient's eye 25, a fixation spot (not referenced additional) is provided at which a patient stare. This fixation spot is generated by a visible lamp 29, collimated by lens 30 introduced into the further optical path 43 of the camera 28 by partial reflector 31.

The treatment laser module 20 and scanner 22 are controlled by a controller 32, while a processor 33 performs the necessary electronic and data processing from the operator, camera 28 and eye-probe system 27. A display 34 provides an operator interface.

Other components common with ophthalmic systems such as viewing binoculars for an operator, illumination slit lamps or aiming beams have not been shown for ease of clarity, however they can be integrated with those components shown in FIG. 4 by anyone skilled in the art of medical laser engineering.

The whole system is able to be translated with respect to an eye 25, in order to focus the beams 21, 38.

Notably the camera 28 focus is a few hundred microns closer than that of the treatment laser 21 and probe beam 38, ensuring that the treatment and probe beams 21 and 38 are focussed below the sclera 2, 11 if the camera 28 is focussed on the sclera 2 respectively 11.

The eye-probe system 27 will now be discussed in more detail as it can take several forms.

Especially the eye probe system 27 or components thereof can realise the present detection system 40 or at least components thereof, or vice versa.

In one form suitable for the embodiment described above, the eye-probe system 27 comprises a photo detector 45 able to detect the reflected beam 38 of a probe beam 38 oh light.

This probe beam 38 of light may be the same as the treatment laser beam 21 or can be a separate light beam optimised for the function.

In order to determine the laser energy of the treatment laser beam 21 required to produce damage of melanin cells of the trabecular meshwork 7, 12 or 13, the treatment laser beam 21 is repeatedly scanned along one path of a radial segment 15 whilst increasing the laser energy until bubble formation is detected.

This threshold power is recorded and stored with a margin, such as 20%, to ensure vaporisation is achieved with other segments. The whole pattern is then scanned with the laser set at the stored energy.

While the above embodiment and method may be functional, it is desirable to better locate the position of the trabecular meshwork 7, 12 resp. 13 in order to minimise the energy delivered to the eye and to minimise the duration of the treatment.

The specific location of a trabecular meshwork 7, 12 or 13 can be located by having especially the eye-probe system 27 include an optical coherence tomography (OCT) system 48. This arrangement represents that of a second preferred embodiment.

The OCT method has been used successfully for determining laser doses in retinal treatments as described in an article in Vol 9, No. 7 of Biomedical Optics Express—“Selective retina therapy enhanced with optical coherence tomography for dosimetry control and monitoring: a proof of concept study” by Daniel Kauffman. The application in that instance is for the retina, with a transparent medium adjacent the layers of interest.

However, in this invention the technique is applied through the sclera 2, 11 that is semi-opaque, and despite both absorption and scattering, a profile of the outer layers of the eye 25, including the TM, can be generated.

OCT provides a few methods of operation, notably a static depth profiling referred to as an A-scan; a traversal along the surface of the object (eye in this instance), referred as a B-scan; and a movie of an A-scan referred to as an M-scan.

It is while performing an M-scan that a change in the reflectivity of a region, for example by the generation of a micro-bubble, can be detected.

OCT systems are commercially available and are based on either a scanning or a spectrometer principle.

For expediency, it is preferred that a spectrometer principle be used for this invention.

In this invention, the profile around the trabecular meshwork 7, 12 resp. 13 is generated by scanning the OCT beam radially outwards about 1 — 2 mm from the sclera-cornea junction. From this profile the trabecular meshwork 7, 12 resp. 13 can be identified and located with good precision and its radial location relative to the outer iris or sclera-cornea boundary can be digitally recorded.

Repeating this exercise at various locations around the eye 25 allows a trabecular meshwork map to be generated by the processor through interpolation of results.

After locating the target for treatment, the probe beam 38 associated with the OCT is positioned at the target and remains there in A-scan mode while the treatment laser 21 beam is activated.

The treatment laser beam 21 delivers pulses of increasing energy until a reaction is detected by the OCT system.

This energy is recorded and used for subsequent deliveries to other regions along the circumference of the trabecular meshwork 7, 12 resp. 13.

An alternative for determining the dose is to maintain the treatment laser beam 21 at a single location focussed on the trabecular meshwork 7, 12 resp. 13 and deliver repeated low energy pulses until a reaction is detected by the OCT in A-scan mode, after which the treatment is paused and the target moves to a next site.

The key to this method is that pulses of energy must be delivered at a rate higher than the relaxation of the cells so that the total energy in the cell increase to the point of micro-bubble formation.

Another alternative is to start at a lower dose energy and deliver repeated pulses of increasing energy to the same location on the trabecular meshwork 7, 12 resp. 13 while monitoring the OCT signal for a change corresponding to micro-bubble formation. When that reaction is achieved, the treatment laser beam 21 is paused and progressed to the next location.

This alternative method works well if the pulse rate is slower than the thermal relaxation of the cells and the cells are able to dissipate the energy from the previous pulse prior to the next pulse arriving.

Both these methods will provide some dose information to use for subsequent locations on the trabecular meshwork 7, 12 resp. 13.

While a pulsed laser is referred to here, a continuous wave (CW) laser may also be used.

The treatment laser beam 21 is of suitable wavelength to be absorbed by the melanin cells, typically green lasers (532 nm) are used for SLT, but longer wavelengths up to 800 nm could be used for better penetration through the sclera 2, 11.

The OCT system 48 would operate in the rage of the infrared wavelength for good transmission through the sclera 2, 11 (800 nm to 1550 nm).

Both the treatment and OCT lasers can be combined into a single module, ensuring their co-linearity during integration into the remainder of the system.

Preferably the imaging camera observes the entire eye in near infra-red, such as 700 nm to 900 nm, light which can be produced by LEDs mounted near the objective lens.

To modulate the energy of the treatment laser beam 21 the apparatus 18 comprises an energy control system 50, which is preferred one part of the detection system 40.

This makes it particularly easy to adjust the expended energy resp. beam energy in relation to detected micro-cavitation, in particular micro-bubbles, and/or in relation to detected location and/or shape, in particular asymmetry, of the trabecular meshwork 7, 12 or 13.The above provides an overview of the essence of the invention without including components or details that are either common in the field or are well understood by engineers in an opto-mechanical field.

### REFERENCES

1 cornea

2 sclera

3 anterior chamber

4 iris

5 lens

6 posterior chamber

7 trabecular meshwork

8 Schlem's canal

9 pupil

10 iris

11 sclera

12 trabecular meshwork

13 trabecular meshwork

14 uncertainty annulus

15 radial lines or radial segment

16 angle

18 ophthalmic apparatus

20 treatment laser module

21 input treatment laser beam

22 2-axis scanner

23 dichroic or partial reflector

24 focusing lens

25 eye

26 reflector

27 eye-probe sub-system

28 camera

29 red lamp

30 lens

31 partial reflector

32 controller

33 processor

34 display

38 probe beam

40 detection system

41 optical path of probe beam

42 treatment laser beam path

43 further optical path

45 photo detector

48 optical coherence tomography (OCT) system

50 energy control system

inner radius R1

outer radius R2

1. An ophthalmic apparatus (18) for treating glaucoma in a patient's eye (25) comprising a treatment laser module (20) delivering a treatment laser beam (21), and comprising a detection system (40) for detecting micro-cavitation, in particular micro-bubbles, formed on account of the treatment laser beam (21) in the patient's eye (25), in particular at the trabecular meshwork (7, 12, 13) of the patient's eye (25).

2. An ophthalmic apparatus (18), in particular according to claim 1, for treating glaucoma in a patient's eye (25) comprising a treatment laser module (20) delivering a treatment laser beam (21), and comprising a detection system (40) for detecting the location and/or the shape, in particular a possible asymmetry, of the trabecular meshwork (7, 12, 13) of the patient's eye (25).

3. An apparatus (18) as in claim 1 or 2, wherein said detection system (40) comprises a tomography system and/or comprises an optical coherence tomography (OCT) system (48) for detecting the location and/or the shape in particular a possible asymmetry, of the trabecular meshwork (7, 12, 13) of the patient's eye (25) and/or the micro-cavitation in particular micro-bubbles.

4. An apparatus (18) as in claim 1, wherein said apparatus (18) comprises an eye-probe sub-system (27) emitting a co-axial probe beam (38).

5. An apparatus (18) as in claim 1, wherein said beams (21, 27A) are focused behind the sclera (2, 11) of a patient's eye (25) in particular through to a trabecular meshwork (7, 12, 13) of a patient's eye (25).

6. An ophthalmic apparatus (18), in particular according to claim 1 or 2, for treating glaucoma comprising a treatment laser module (20) delivering a treatment laser beam (21) to a scanner (22) and an objective focusing lens (24), and comprising a co-axial probe beam (38) emitted from an eye-probe sub-system (27), said beams (21, 38) focused behind the sclera (2, 11) and through to a trabecular meshwork (7, 12, 13) of a patient's eye (25), the apparatus (18) including a detector (45) within the eye-probe sub-system (27) that senses backscattered light from the probe beam (38) and detects the formation of micro-bubbles formed on account of the treatment laser beam (21) inducing damage to the melanin cells in the trabecular meshwork (7, 12, 13).

7. An apparatus (18) as in claim 1, wherein said apparatus (18) comprises an energy control system (50), which modulates the treatment laser beam (21) in dependence of information of the detection system (40).

8. An apparatus (18) as in claim 1, wherein an eye-probe sub-system (27) comprises an optical coherence tomography (OCT) system that further determines the location of the trabecular meshwork (7, 12, 13) prior to delivery of the treatment laser beam (21).

9. An apparatus (18) as in claim 1, wherein an eye-probe sub-system (27) comprises a photo-detector (45).

10. An apparatus (18) as in claim 1, wherein the probe beam (38) is represented by the treatment laser beam (21).

11. An apparatus (18) as in claim 1, wherein the wavelength of the treatment laser beam (21) is in the absorption range of melanin cells and the probe beam (38) is infra-red.

12. A method of treating glaucoma characterised by determining through a sclera (2, 11) the location and/or the shape in particular a possibly asymmetry of a trabecular meshwork (7, 12, 13) and delivering a treatment laser beam (21) to that location with a beam energy sufficient to generate micro-bubbles.

13. A method of treating glaucoma as in claim 12, whereby the energy of the treatment laser beam (21) is controlled and adjusted depending on the effect of micro-bubbles or micro-cavitation, and/or of the location and or the shape in particular a possible asymmetry of the trabecular meshwork (7, 12, 13).

14. A method of treating glaucoma as in claim 12 or 13, whereby an optical coherence tomography (OCT) system (48) is firstly used to identify the location of the trabecular meshwork (7, 12, 13), whereupon the treatment laser beam (21) is directed at that location and either a pre-set laser energy dose is delivered to the location or the energy dose is increased until the optical coherence tomography (OCT) system (48) detects micro-bubbles.

15. A method as in claim 12, whereby the beams follow a pattern in accordance with inputs from an energy control system (50), in particular a processor (33) and controller (32).

16. A method as in claim 12, wherein the pattern comprises radial lines (15) extending from an inner radius (R1) to an outer radius (R2), the radii corresponding to extremes of the likely position of a trabecular meshwork (7, 12, 13).
